(19) United States
(12) Patent Application Publication
(10) Pub. No.: US 2013/0211464 A1
(43) Pub. Date: Aug. 15, 2013

(54) LOCK PIN TYPE CENTRAL NAIL DEVICE

(71) Applicants: Zhixiong Lin, Xiamen (CN); Xigui Zhang, Xiamen (CN)

(72) Inventors: Zhixiong Lin, Xiamen (CN); Xigui Zhang, Xiamen (CN)

(73) Assignee: XIAMEN DOUBLE ENGINE MEDICAL MATERIAL CO., LTD, Xiamen, Fujian (CN)

(21) Appl. No.: 13/818,482

(22) PCT Filed: Oct. 26, 2011
(86) PCT No.: PCT/CN2011/081304

§ 371 Date: Apr. 8, 2013

(30) Foreign Application Priority Data
Jun. 23, 2011 (CN) 201120217489.X

(51) Int. Cl. A61B 17/86

(52) U.S. Cl. CPC A61B 17/8605
USPC 606/304

(57) ABSTRACT

A lock pin type central nail device has a hollow screw and a central nail, the external revolution surface of the hollow screw is provided with a first external thread segment, a central hole is arranged in the hollow screw in a axially penetrating manner, and the central nail is adaptively mounted into the central hole of the hollow screw, so that the hollow screw and the central nail (20) are mounted together.

### FIELD OF THE INVENTION

The present invention relates to a lock pin type central nail device.

### BACKGROUND OF THE INVENTION

The hollow screw and its application method for treating fracture and dislocation at the prior comprises: the tack is is drive into bone first, then the hollow screw is sleeved to the tack pin, and then the hollow screw is drive into bone along the tack pin, and then the tack pin is taken out. In the said fixing nail device, only the hollow screw plays a fixed role in essence, so the defects of the said fixing nail device are: 1 the strength is poor, and the pin is easy to be bent or broken, leading to the failure of the internal fixing; 2 the hollow screw is easy to be distorted, leading to the failure of the internal fixing.

### SUMMARY OF THE INVENTION

The object of the present invention is to offer a well-designed-and-developed lock pin type central nail device which overcomes the defect of the central nail at the prior.

The technical proposal solving the technical matter in the present invention is:

A lock pin type central nail device comprises a hollow screw (10) and a central nail (20), the external revolution surface of the hollow screw (10) is provided with a first external thread segment (11), a central hole (12) is arranged in the hollow screw (10) in a axially penetrating manner, and the central nail (20) is adaptively mounted into the central hole (12) of the hollow screw (10), so that the hollow screw (10) and the central nail (20) are mounted together.

In a preferred embodiment, the internal revolution surface of the central hole (12) of the hollow screw (10) is provided with a internal thread segment (13), the external revolution surface of the central nail (20) is provided with a second external thread segment (21), the internal thread segment (13) of the central hole (12) and the second external thread segment (21) are adaptively screwed together.

In a preferred embodiment, a broaching (12a) is arranged at the back end of the central hole (12) of the hollow screw (10), and a connector (22) is arranged at the central nail (20), and the broaching (12a) is adaptive to the connector (22), and a plug slot (22a) is concavely arranged on the end face of the connector (22).

In a preferred embodiment, the first external thread segment (11) is arranged at the front end of the hollow screw (10); the internal thread segment (13) is arranged near the broaching (12a).

In a preferred embodiment, a ring groove (14) is concavely arranged on the internal revolution surface of the central hole (12) of the hollow screw (10), a ring flange (23) adaptive to the ring groove (14) is convexly arranged on the external revolution surface of the central nail (20); and at least two kerf slots (24) that radially penetrates and intersects are concavely arranged on the end face of the central nail (20), and the axial depth of the kerf slot (24) is bigger than the maximum interval (d) between the end face of the central nail (20) and the ring flange (23).

In a preferred embodiment, the front end face of the ring flange (23) is designed to be a conical surface with small front and big back and the back end face of the ring flange (23) is designed to be a circular surface.

In a preferred embodiment, the two kerf slots (24) intersect to be a cross.

In a preferred embodiment, a broaching (12a) is arranged at the back end of the central hole (12), the back end of the central nail (20) is convex to be a connector (22) adaptive to the broaching (12a).

In a preferred embodiment, the front end face of the broaching (12a) is designed to be a conical surface with small front and big back, and the front end face of the connector (22) is arranged to be a conical surface adaptive to the front end face of the broaching (12a).

In a preferred embodiment, the hollow screw (10) is planted into the central position, and then the central nail (20) is adaptively mounted into the central hole (12) of the hollow screw (10), so that the hollow screw (10) and the central nail (20) are mounted and locked automatically together.

The lock pin type central nail device in the present invention is mainly used for treating all kinds of fracture and dislocation. Compared with the technical proposal at the prior, the benefits of the present invention are:

1 the central nail is adaptively mounted into the central hole of the hollow screw to mount the hollow screw and the central nail together, the strength of the hollow screw is enhanced, and it prevents the hollow from bending and being out of shape, and the treating operation is easy and fast without increased treating difficulty;

2 the internal revolution surface of the central hole of the hollow screw is provided with an internal thread segment, the external revolution surface of the central nail is provided with a second external thread segment, the internal thread segment of the central hole and the second external thread segment are adaptively screwed together, so the operation is easy and fast, the separation of the hollow screw and the central nail is avoided, and the strength of the hollow screw is enhanced;

3 a broaching is arranged at the back end of the central hole of the hollow screw, and a connector is arranged at the central nail, and the broaching is adaptive to the connector, so that the strength of the hollow screw is enhanced; and a plug slot is concavely arranged on the end face of the connector, so that it is easy to screw the central nail into the hollow screw;

4 the internal thread segment of the central hole is arranged near the broaching, so that it is easy to screw the central nail into the hollow screw;

5 a ring groove is arranged at the central hole of the hollow screw, and a ring flange is convexly arranged at the central nail, and the kerf slots are arranged on the end face of the central nail, so that the back part of the central nail is provided with elasticity and is able to contract inward and expand outward, so the ring flange is easy to be mounted into the ring groove, the connection is firm and reliable, and the strength of the hollow screw is enhanced;

6 the front end face of the ring flange is designed to be a conical surface with small front and big back to play the role of orientation, and the back end face of the ring flange is designed to be a circular surface to prevent from pulling out;

7 the front end face of the broaching is designed to be a conical surface with small front and big back, and the front end face of the connector is designed to be a conical surface adaptive to the front end face of the broaching, so that the broaching of the hollow screw is provided with a elastic edge, and it is easy to mount the central nail into the hollow screw;

### BRIEF DESCRIPTION OF THE DRAWINGS

FIG. 1 shows the structure schematic view of the lock pin type central nail device in a preferred embodiment;

FIG. 2 shows the structure schematic view of the hollow screw of the lock pin type central nail device in a preferred embodiment;

FIG. 3 shows the structure schematic view of the central nail of the lock pin type central nail device in a preferred embodiment;

FIG. 4 shows the amplification schematic view of FIG. 1A;

FIG. 5 shows the structure schematic view of the lock pin type central nail device in another preferred embodiment;

FIG. 6 shows the structure schematic view of the hollow screw of the lock pin type central nail device in another preferred embodiment;

FIG. 7 shows the structure schematic view of the central nail of the lock pin type central nail device in another preferred embodiment;

FIG. 8 shows the amplification schematic view of FIG. 5B.

### DESCRIPTION OF THE PREFERRED EMBODIMENTS

With the following description of the drawings and specific embodiments, the invention shall be further described in details.

### Embodiment 1

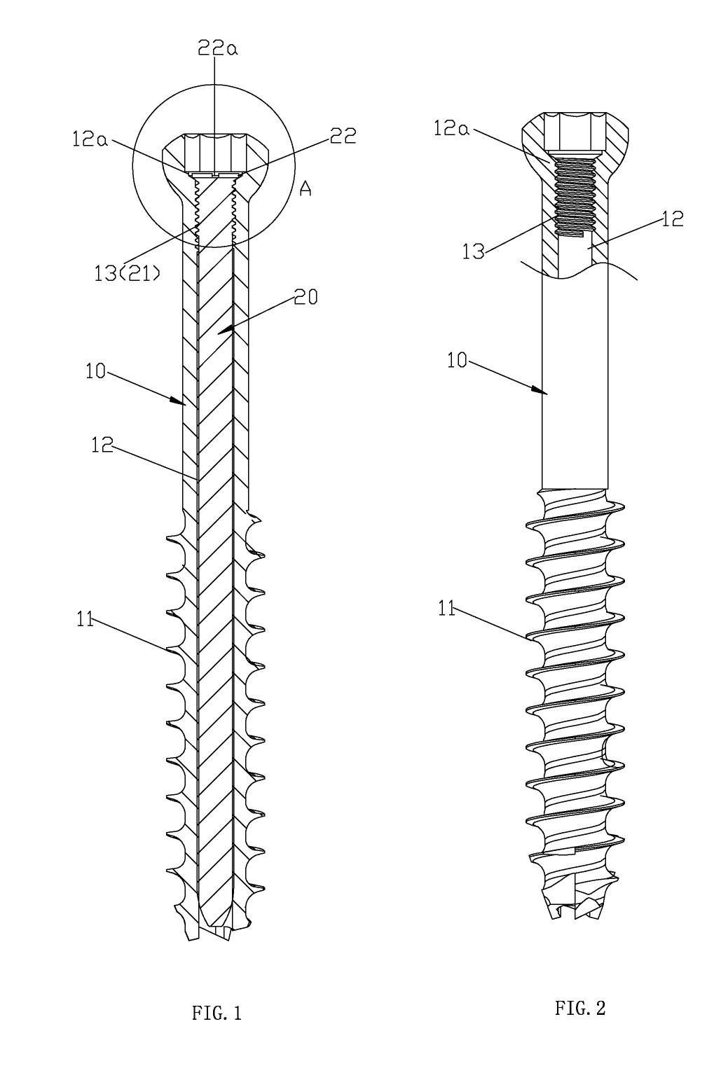

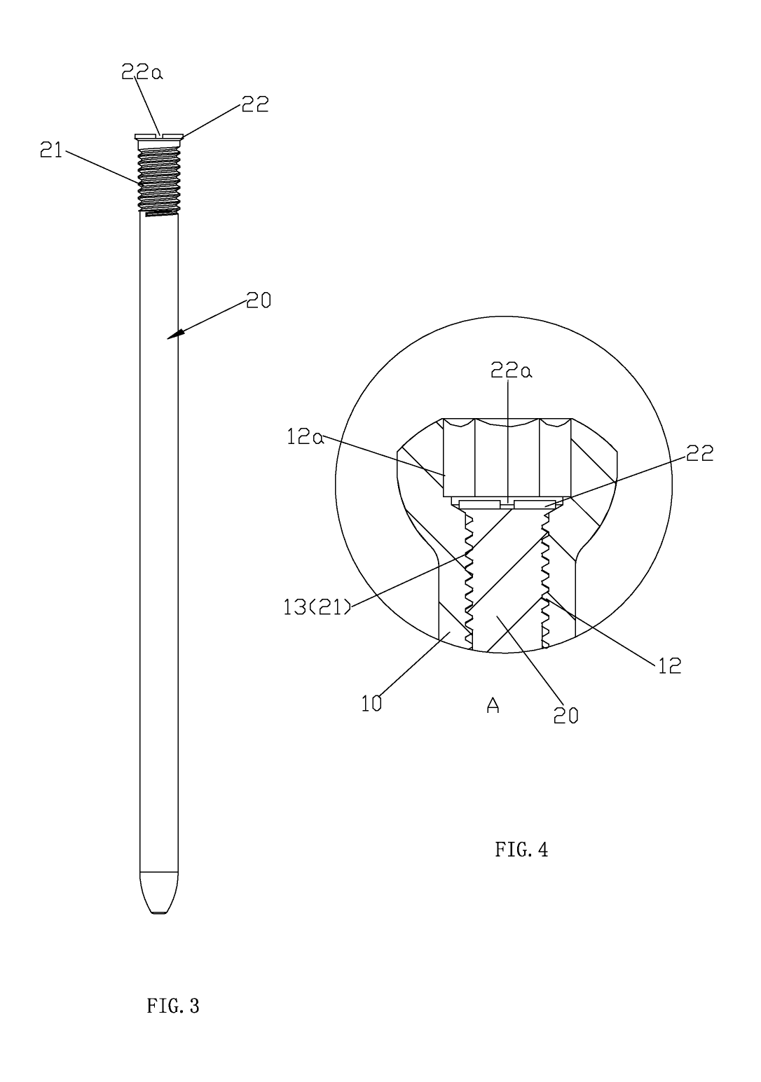

According to FIG. 1 to 4, a lock pin type central nail device comprises a hollow screw 10 and a central nail 20.

The external revolution surface of the hollow screw 10 is provided with a first external thread segment 11, and in the present embodiment, the first external thread segment 11 is arranged at the front end of the hollow screw 10. A central hole 12 is arranged in the hollow screw 10 in a axially penetrating manner. A broaching 12a is arranged at the back end of the central hole 12, and the internal revolution surface of the central hole 12 is provided with a internal thread segment 13 which is arranged near the broaching 12a.

The external revolution surface of the central nail 20 is provided with a second external thread segment 21 which is adaptive to the internal thread segment 13 of the central hole 12. A connector 22 which is adaptive to the broaching 12a is arranged at the back end of the central nail 20. And a plug slot 22a is concavely arranged on the end face of the connector 22.

The central nail 20 is adaptively mounted into the central hole 12 of the hollow screw 10, and the second external thread segment 21 of the central nail 20 is adaptively screwed with the internal thread segment 13 of the central hole 12, so that the hollow screw 10 and the central nail 20 are mounted together.

In the present lock pin type central nail device, the central nail is screwed into the hollow screw through the external screw of the central nail to combined the external screw of the central nail with the internal screw of the tail of the hollow screw, so that the hollow screw and the central nail form a whole with enhanced hollow screw strength, ideal and strong internal fixed pattern and reduced risk of crooked or broken hollow screw.

### Embodiment 2

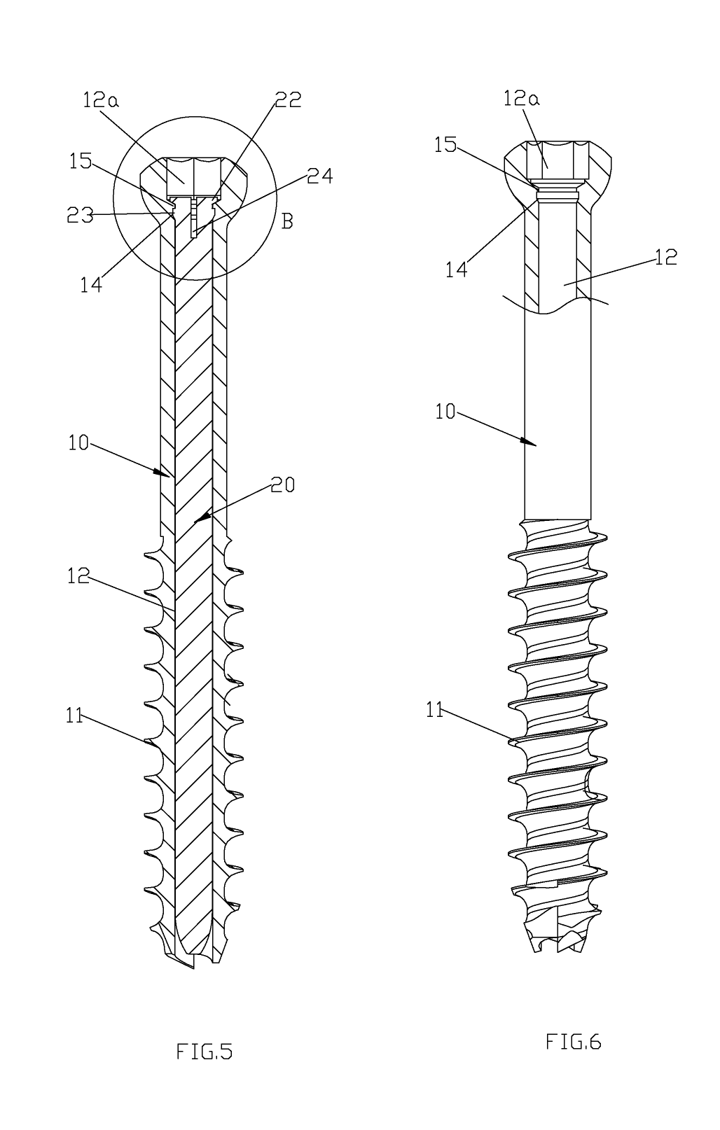

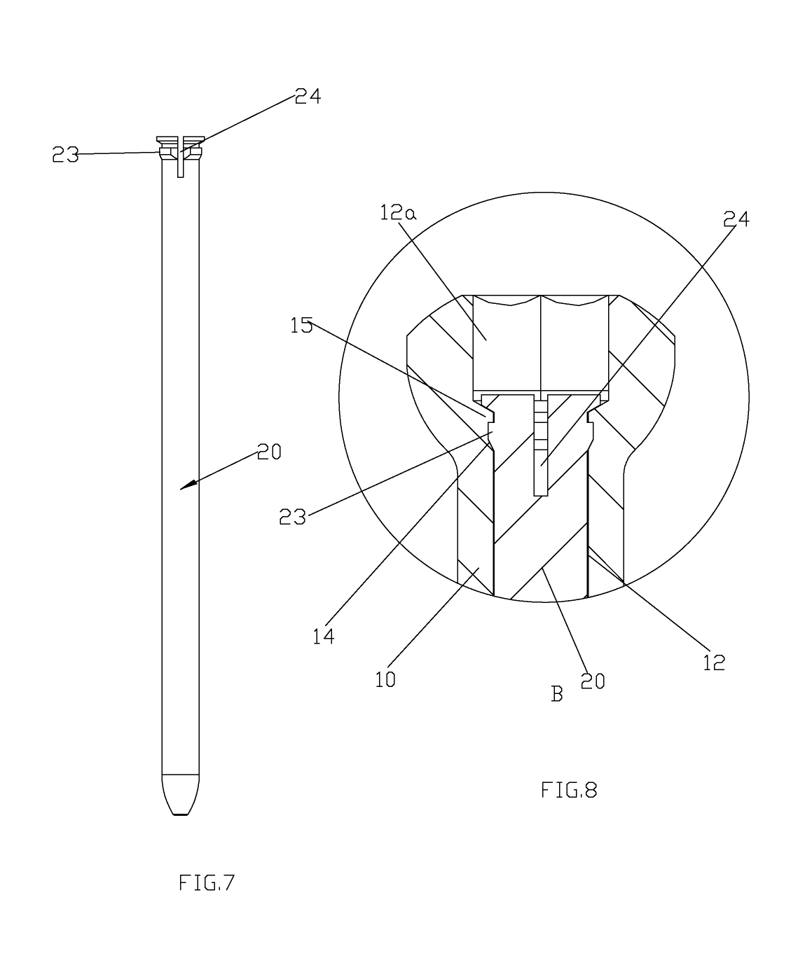

According to FIG. 5 to 8, the difference from the embodiment 1 is: a ring groove 14 of which the front end face is designed to be a conical surface with small front and big back and the back end face is designed to be a circular surface is concavely arranged on the internal revolution surface of the central hole 12. A broaching 12a of which the front end face is designed to be a conical surface with small front and big back is arranged at the back end of the central hole 12 of the hollow screw 10.

A ring flange 23 adaptive to the ring groove 14 is concavely arranged on the external revolution surface of the central nail 20, of which the front end face is designed to be a conical surface with small front and big back and the back end face is designed to be a circular surface. The back end of the central nail 20 is convex to be a connector 22 adaptive to the broaching 12a, and front end face of the connector 22 is designed to be a conical surface adaptive to the front end face of the broaching 12a, so that the broaching of the hollow screw is provided with a elastic edge 15. And at least two kerf slots 24 that radially penetrates and intersects are concavely arranged on the end face of the central nail 20, and the axial depth of the kerf slot 24 is bigger than the maximum interval d between the end face of the central nail 20 and the ring flange 23. In the present embodiment, the two kerf slots 24 intersect to be a cross.

The central nail 20 is adaptively mounted into the central hole 12 of the hollow screw 10, and the ring flange 23 is adaptively clamped into the ring groove 14, so that the hollow screw 10 and the central nail 20 are mounted together.

In the present groove type and lock pin type central nail device, the step design of the central nail and the elastic groove design are used, the central nail is inserted into the hollow screw, and then the step of the tail of the central nail encounter the resistance of the internal wall of the hollow screw, and then the tail of the central nail contracts automatically, after the central nail is completely inserted into the hollow screw, the tail of the central nail is opened again, so that the central nail is fixed in the groove of the internal wall of the hollow screw: the hollow screw and the central nail form a whole with enhanced hollow screw strength, ideal and strong internal fixed pattern and reduced risk of crooked or broken hollow screw.

The lock pin type central nail device in the present invention is provided with the following benefits: 1 after the hollow screw is planted in the central position, the central nail is locked into the hollow screw, and the strength of the hollow screw is enhanced, and the crooking or breaking of the hollow screw is prevented; 2 the central nail is locked into the hollow screw, it can lock automatically to prevent the central nail from dropping out; 3 the central nail does not affect the function of the hollow screw when it is enhancing the strength of the hollow screw; 4 it is designed to be bolt type, and it is convenient to operate.

The invention has been described with reference to the preferred embodiments mentioned above; therefore it cannot limit the reference implementation of the invention, it is obvious to a person skilled in the art that structural modification and changes can be carried out without leaving the scope of the claims hereinafter and the description above.

### INDUSTRIAL APPLICABILITY

The lock pin type central nail device is described in the present invention, the central nail is adaptively mounted into the central hole of the hollow screw, when it is actual used: 1 after the hollow screw is planted in the central position, the central nail is locked into the hollow screw, and the strength of the hollow screw is enhanced, and the crooking or breaking of the hollow screw is prevented; 2 the central nail is locked into the hollow screw, it can lock automatically to prevent the central nail from dropping out; 3 the central nail does not affect the function of the hollow screw when it is enhancing the strength of the hollow screw; 4 it is designed to be bolt type, and it is convenient to operate.

What is claimed is:

1. A lock pin type central nail device, wherein, it comprises a hollow screw and a central nail, the external revolution surface of the hollow screw is provided with a first external thread segment, a central hole is arranged in the hollow screw in a axially penetrating manner, and the central nail is adaptively mounted into the central hole of the hollow screw, so that the hollow screw and the central nail are mounted together.

2. A lock pin type central nail device according to claim 1, wherein, the internal revolution surface of the central hole of the hollow screw is provided with a internal thread segment, the external revolution surface of the central nail is provided with a second external thread segment, the internal thread segment of the central hole and the second external thread segment are adaptively screwed together.

3. A lock pin type central nail device according to claim 2, wherein, a broaching is arranged at the back end of the central hole of the hollow screw, and a connector is arranged at the central nail, and the broaching is adaptive to the connector, and a plug slot is concavely arranged on the end face of the connector.

4. A lock pin type central nail device according to claim 3, wherein, the first external thread segment is arranged at the front end of the hollow screw; the internal thread segment is arranged near the broaching.

5. A lock pin type central nail device according to claim 1, wherein, a ring groove is concavely arranged on the internal revolution surface of the central hole of the hollow screw, a ring flange adaptive to the ring groove is convexly arranged on the external revolution surface of the central nail; and at least two kerf slots that radially penetrates and intersects are concavely arranged on the end face of the central nail, and the axial depth of the kerf slot is bigger than the maximum interval (d) between the end face of the central nail and the ring flange.

6. A lock pin type central nail device according to claim 5, wherein, the front end face of the ring flange is designed to be a conical surface with small front and big back and the back end face of the ring flange is designed to be a circular surface.

7. A lock pin type central nail device according to claim 5, wherein, the two kerf slots intersect to be a cross.

8. A lock pin type central nail device according to claim 5, wherein, a broaching is arranged at the back end of the central hole, the back end of the central nail is convex to be a connector adaptive to the broaching.

9. A lock pin type central nail device according to claim 8, wherein, the front end face of the broaching is designed to be a conical surface with small front and big back, and the front end face of the connector is arranged to be a conical surface adaptive to the front end face of the broaching.

10. A lock pin type central nail device according to claim 2, wherein, the hollow screw is planted into the central position, and then the central nail is adaptively mounted into the central hole of the hollow screw, so that the hollow screw and the central nail are mounted and locked automatically together.

11. A lock pin type central nail device according to claim 6, wherein, the two kerf slots intersect to be a cross.

12. A lock pin type central nail device according to claim 6, wherein, a broaching is arranged at the back end of the central hole, the back end of the central nail is convex to be a connector adaptive to the broaching.

13. A lock pin type central nail device according to claim 8, wherein, the front end face of the broaching is designed to be a conical surface with small front and big back, and the front end face of the connector is arranged to be a conical surface adaptive to the front end face of the broaching.

14. A lock pin type central nail device according to claim 5, wherein, the hollow screw is planted into the central position, and then the central nail is adaptively mounted into the central hole of the hollow screw, so that the hollow screw and the central nail are mounted and locked automatically together.
